(19) United States
(12) Patent Application Publication
(10) Pub. No.: US 2024/0019932 A1
(43) Pub. Date: Jan. 18, 2024

(54) PROGRAM, INFORMATION PROCESSING METHOD, AND INFORMATION PROCESSING APPARATUS

(71) Applicants: VIE STYLE, INC., Kanagawa (JP); NTT DATA INSTITUTE OF MANAGEMENT CONSULTING, INC., Tokyo (JP)

(72) Inventors: Yasuhiko IMAMURA, Kanagawa (JP); Takuya IBARAKI, Tokyo (JP)

(21) Appl. No.: 18/037,722

(22) PCT Filed: Nov. 19, 2020
(86) PCT No.: PCT/JP2020/043280

§ 371 Date: Sep. 27, 2023

(51) Int. Cl. G06F 3/01; A61B 5/291; A61B 5/00

(52) U.S. Cl. CPC G06F 3/015; A61B 5/291; A61B 5/6817

(57) ABSTRACT

EEG to be measured on a scalp is estimated based on EEG measured from an ear canal. The program causes a processor included in an information processing apparatus to execute: acquiring first EEG information that is measured by a first bio-electrode that comes into contact with an ear canal of a predetermined user; acquiring second EEG information that is measured by a brain activity measuring instrument with respect to the predetermined user at the same timing as the first EEG information; learning a relationship between a first feature of the first EEG information and a second feature of the second EEG information; and generating a learning model that has learned the relationship.

### TECHNICAL FIELD

The present invention relates to a program, an information processing method, and an information processing apparatus.

### BACKGROUND ART

Heretofore, earphones for acquiring electroencephalogram signals are known (see Patent Document 1, for example).

### CITATION LIST

Patent Document
Patent Document 1: Patent Publication JP-A-2018-159908

### SUMMARY

### Technical Problem

When acquiring an electroencephalogram signal from an earphone, the electroencephalogram signal is acquired from two electrodes that respectively come into contact with left and right ear canals. Therefore, compared with the ten-twenty electrode system of the International Federation for measuring based on a head outer shape that is typically used as the arrangement of electrodes, the signal source provided in the earphone is limited.

Here, when the brain function is predicted based on electroencephalogram (EEG), the brain function is estimated by predicting excited states of regions, based on voltages. For example, it is known that perception or the like is related to the prefrontal area of the cerebral neocortex, and visual recognition is related to the lobus parietalis and the lobus occipitalis. Also, it is known that the lobus temporalis close to ear canals is related to the hearing sense and sound/language recognition. Therefore, with the EEG acquired from ear canals by an earphone, signals related to the hearing sense and sound can be acquired at a high quality, but signals associated with other regions such as the visual sense and conception cannot be acquired at a high quality.

Therefore, one aspect of the present invention aims at providing a program, an information processing method, and an information processing apparatus that enable, by finding out the relationship between EEG measured from ear canals and EEG measured by a brain activity measuring instrument and using this relationship, estimation of the EEG to be measured by the brain activity measuring instrument, based on the EEG measured from ear canals.

### Solution to Problem

A program according to one aspect of the present invention is for causing a processor included in an information processing apparatus to execute: acquiring first EEG information that is measured by a first bio-electrode that comes into contact with an ear canal of a predetermined user; acquiring second EEG information that is measured by a brain activity measuring instrument with respect to the predetermined user at the same timing as the first EEG information; learning a relationship between a first feature of the first EEG information and a second feature of the second EEG information; and generating a learning model that has learned the relationship.

### Advantageous Effects of Invention

According to one aspect of the present invention, the brain activity to be measured by a brain activity measuring instrument can be estimated based on a brain activity measured from ear canals.

### BRIEF DESCRIPTION OF DRAWINGS

FIG. 1 is a diagram for describing an exemplary outline of a system according to an embodiment.

FIG. 2 is a diagram illustrating an example of positions of scalp electrodes according to the embodiment.

FIG. 3 is a diagram illustrating an example of an earphone set according to the embodiment.

FIG. 4 is a diagram illustrating an example of a schematic cross section of the earphone according to the embodiment.

FIG. 5 is a diagram illustrating estimation accuracy when learning EEG signals on a scalp.

FIG. 6 is a diagram illustrating estimation accuracy when learning EEG signals of an ear canal.

FIG. 7 is a diagram illustrating an example of a model of the EEG signal in an ear canal.

FIG. 8 is a diagram illustrating an example of a model of the EEG signal on a scalp.

FIG. 9 is a diagram illustrating an example of a model of an estimated EEG signal on a scalp.

FIG. 10 is a diagram illustrating estimation accuracy when learning estimated EEG signals on a scalp.

FIG. 11 is a block diagram illustrating an example of an information processing apparatus according to the embodiment.

FIG. 12 is a block diagram illustrating an example of an information processing apparatus according to the embodiment.

FIG. 13 is a flowchart illustrating an example of model generation processing of a server according to the embodiment.

FIG. 14 is a flowchart illustrating an example of learning processing according to the embodiment.

FIG. 15 is a flowchart illustrating an example of brain activity estimation processing according to the embodiment.

FIG. 16 is a flowchart illustrating an example of command operation processing according to the embodiment.

### DESCRIPTION OF EMBODIMENTS

The following describes an embodiment of the present invention with reference to the drawings. Note that the embodiment described below is merely illustrative, and is not intended to exclude any modifications and technical applications that are not explicitly described below. In other words, the present invention can be carried out with various modifications without departing from the spirit of the invention. Also, in the drawings referenced below, identical or similar elements are denoted by identical or similar reference numerals. The drawings are schematic, and the illustrations therein do not necessarily coincide with the actual dimensions, ratios, and the like. Also, the dimensional relationships and ratios of portions may differ between drawings.

### EMBODIMENT

An outline of a system in the embodiment will be described below using the drawings.

<Outline of System>

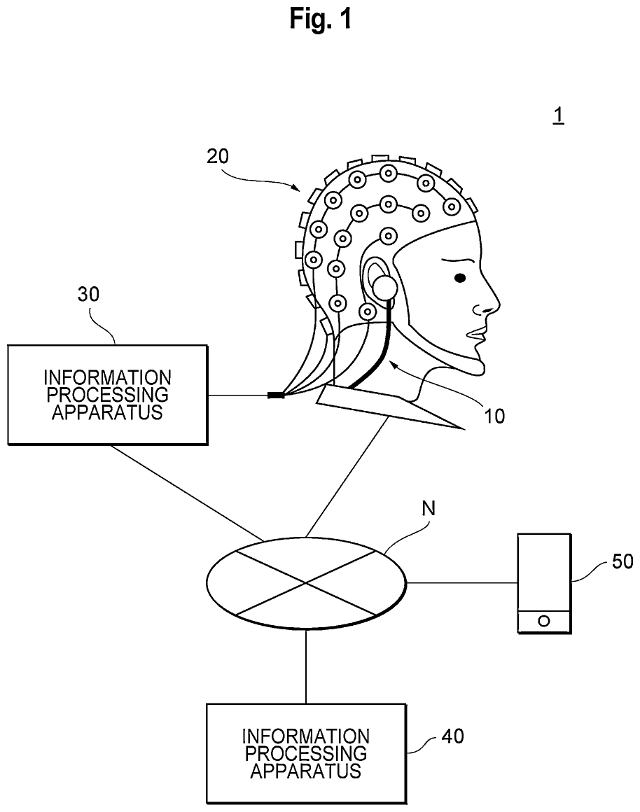

First, an exemplary outline of a system 1 according to the embodiment will be described using FIG. 1. In the system 1, a user for which EEG will be measured wears an earphone set 10 that provides electrodes to ear canals. In the example in FIG. 1, a neck hanging type earphone set 10 is illustrated, but any earphone may also be used as long as an EEG signal (first EEG signal) can be sensed from ear canals. For example, an earphone set that acquires a reference signal from an earlobe, an earphone that acquires a reference signal or an earth signal from another position (another position in the ear canal), and a completely wireless earphone can be used.

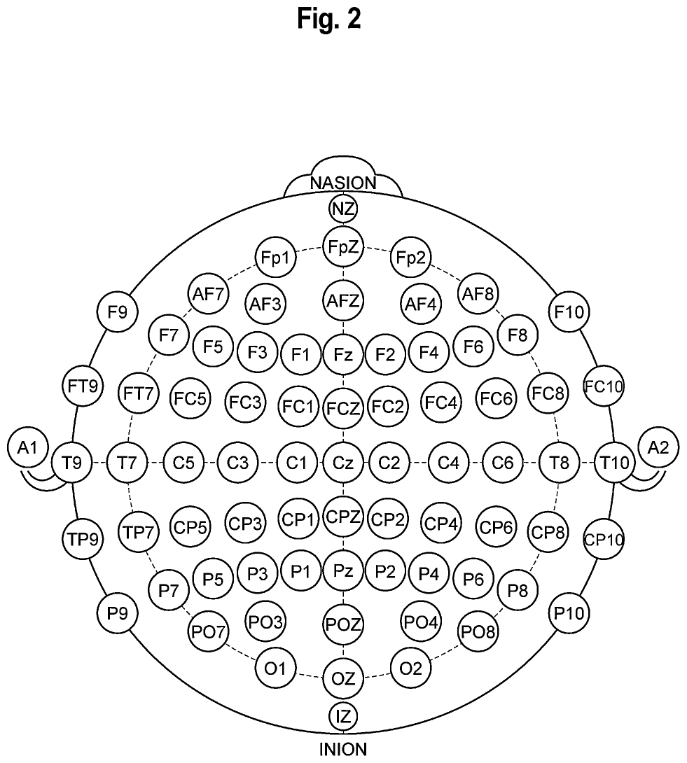

Also, the user wears a headgear 20 that measures EEG using the ten-twenty electrode system of the International Federation, for example. The headgear 20 is an example of a brain activity measuring instrument. In the headgear 20, electrodes are provided at predetermined positions of a scalp, based on the ten-twenty electrode system of the International Federation. FIG. 2 is a diagram illustrating an example of positions of scalp electrodes according to the embodiment. For example, electrodes are provided at front polar (Fp1, Fp2), frontal (F3, F4), anterior-temporal (F7, F8), central (C3, C4), parietal (P3, P4), occipital (O1, O2), mid-temporal (T3, T4), posterior-temporal (T5, T6), auricular (A1, A2), midline frontal (Fz), vertex (Cz), midline parietal (Pz), and the like. The example shown in FIG. 2 is merely an example, and the number of electrodes may also be decreased and increased, as needed. Note that the number of electrodes provided on a scalp is larger than the number of electrodes provided in ear canals.

An information processing apparatus 30 to be connected to the headgear 20 is a measurement apparatus, for example, acquires an EEG signal (second EEG signal) measured on a scalp (or cucullus) to be measured, and performs collection, analysis, output, and the like, as needed. For example, the information processing apparatus 30 may transmit EEG signals of regions for which signal acquisition is performed to an information processing apparatus 40 via a network N.

Also, the earphone set 10 acquires a first EEG signal from ear canals at a timing that is the same as the timing at which a second EEG signal is measured on a scalp, and transmits the first EEG signal to the information processing apparatus 40 via the network N.

The information processing apparatus 40 is a server, for example, and analyzes and extracts a relationship between the second EEG signal on a scalp and the first EEG signal in ear canals that are measured at the same timing. For example, the information processing apparatus 40 extracts a relationship between a first feature of the first EEG signal and a second feature of the second EEG signal. As a specific example, the information processing apparatus 40 extracts features of the first EEG signal and the second EEG signal by performing learning regarding these signals, using a predetermined learning model, and learns the relationship between the features. In this case, the information processing apparatus 40 may transmit the learning model obtained as a result of learning to the information processing apparatus 50. Also, the information processing apparatus 30 and the information processing apparatus 40 may be the same apparatus.

An information processing apparatus 50 is a processing terminal such as a mobile terminal held by a user, for example, and receives a user operation using an EEG signal acquired from the earphone set 10. For example, the information processing apparatus 50 acquires a learning model from the information processing apparatus 40, and estimates an EEG signal to be measured by a brain activity measuring instrument from EEG signals that are successively acquired in the ear canals using this learning model. The information processing apparatus 50 further estimates brain activity information of the user (what the user is thinking, and the like) using the estimated EEG signal, and executes processing corresponding to the estimated brain activity. That is, the information processing apparatus 50 includes an interface for performing operations of a device based on an EEG signal.

Accordingly, even if the number of electrodes is limited due to the narrowness of ear canals, and the features of an EEG signal are constrained depending on the acquisition positions, an EEG signal can be estimated with which a wide range of brain information can be acquired, from EEG signals measured in the ear canals, and can perform prediction of the brain activity using the estimated EEG signal on the scalp, and the like.

<Configuration of Earphone Set>

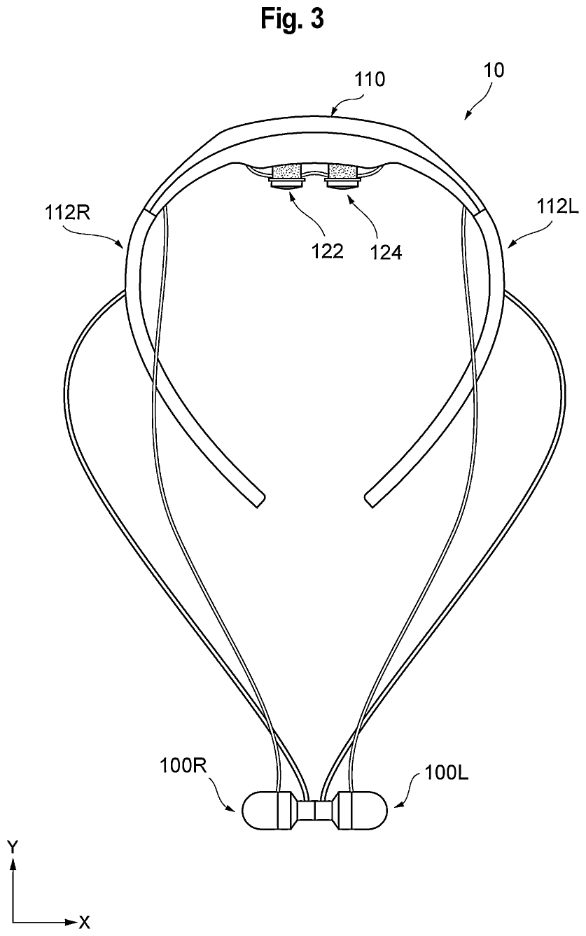

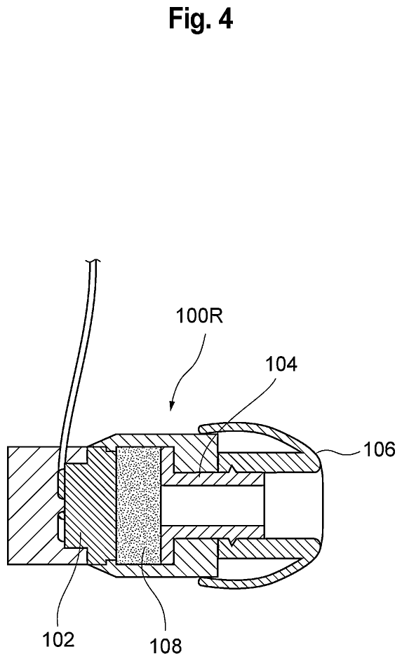

FIGS. 3 and 4 illustrate an outline of the earphone set 10 in the embodiment. Note that the earphone set 10 is not limited to the example shown in FIGS. 3 and 4, and the technique of this disclosure can be applied to any earphone, as long as the earphone can sense EEG from ear canals and output the EEG to an external apparatus.

FIG. 3 is a diagram illustrating an example of the earphone set 10 according to the embodiment. The earphone set 10 shown in FIG. 3 includes a pair of earphones 100R and 100L and a neck hanging portion 110. The earphones 100R and 100L are each connected to the neck hanging portion 110 using a cable through which signals can be communicated, but may also be connected using wireless communication. When left and right do not need to be distinguished, R and L are omitted below.

The neck hanging portion 110 includes a central member that will extend behind a neck and bar-shaped members (arms) 112R and 112L having curved shapes along two sides of the neck. Electrodes 122 and 124 for sensing EEG signals are provided on a surface of the central member that comes into contact with the neck on a back side. The electrodes 122 and 124 each include an electrode to be grounded and a reference electrode. With this, as described below, the electrodes 122 and 124 can be separated from elastic electrodes provided in the ear chips of the earphone, and therefore EEG signals can be accurately acquired. Also, the neck hanging portion 110 may include a processing unit that processes EEG signals and a communication device that communicates with an external apparatus, but the processing unit and communication unit may also be provided in the earphones 100.

Also, in each of the bar-shaped members 112R and 112L on the two sides of the neck hanging portion 110, the portion on a leading end side is heavier than the portion on a base end side (central member side), and accordingly, the electrodes 122 and 124 come in close contact with the neck of a wearer with an appropriate pressure being applied. For example, a weight is provided on the leading end side of each of the bar-shaped members 112R and 112L. Note that the positions of the electrodes 122 and 124 are not limited to these positions.

FIG. 4 is a diagram illustrating an example of a schematic cross section of the earphone 100R according to the embodiment. In the earphone 100R shown in FIG. 4, an elastic member (e.g., urethan) 108 may be provided between a speaker 102 and a nozzle 104, for example. As a result of providing an elastic member 108, the vibration of the speaker 102 is unlikely to propagate to an elastic electrode of the ear chip 106, and the elastic electrode of the ear chip 106 can be prevented from interfering with the speaker 102 regarding sounds.

Moreover, although the ear chip 106 including the elastic electrode is located in a sound introduction port, interference of sound vibration can be prevented due to the elasticity of the elastic electrode. Also, by adopting an elastic member for a housing, sound vibration is unlikely to propagate to the elastic electrode of the ear chip 106 due to this elastic member, and therefore interference of sound vibration can be prevented.

The earphone 100 includes an audio sound processor, and a sound signal with a frequency less than or equal to a predetermined frequency (e.g., 50 Hz) corresponding to the EEG signal may be cut off using this audio sound processor. In particular, the audio sound processor may cut off a sound signal with a frequency less than or equal to 30 Hz, which is in a frequency band in which features are likely to appear as the EEG signal, but may amplify a sound signal with a frequency around 70 Hz in order not to damage a base sound.

Accordingly, the sound signal can be prevented from interfering with the EEG signal. Also, the audio sound processor may cut off a signal with a predetermined frequency only when EEG signals are sensed.

Also, the ear chip 106 conducts an EEG signal obtained by sensing an ear canal to a contact of an electrode provided in the nozzle 104. The EEG signal is transmitted from the ear chip 106 to a biosensor (not shown) inside the earphone 100 via the contact. The biosensor outputs the EEG signals acquired successively to a processing apparatus provided in the neck hanging portion 110 through a cable, or transmits the EEG signals to an external apparatus.

<Outline of Experiments>

Next, experiments performed by the inventors will be described. By the experiments, the relationship between features of EEG signals measured from ear canals and features of EEG signals measured from a scalp has been studied.

First, as shown in FIG. 1, at the same time when an EEG signal is measured from ear canals, an EEG signal is also measured on a scalp. Accordingly, from the EEG signals acquired at the same time, whether or not the EEG signals are related, and the like, can be studied.

Also, the acquired EEG signals are divided into unit time (e.g., 4 seconds) windows, and are transformed into frequency power spectra by performing Fourier transformation for each unit time. These windows are shifted by any interval (e.g., seconds), and the aforementioned transformation is repeatedly performed.

Using the power spectra obtained by transforming the EEG signals, the relationship between features of an EEG signal in the ear canals and features of an EEG signal on the scalp, the EEG signals having been measured at the same time, is learned using machine learning, for example.

An example of a detained learning method is as follows.
(1) A target EEG signal (scalp EEG signal) to be ultimately acquired is denoted as A. A is prepared as a matrix (vector) of Na electrodes×Da dimensions (number of dimensions of specific frequencies).
(2) An EEG signal in ear canals is denoted as B. Similarly to A, B is prepared as a matrix (vector) of Nb electrodes×Db dimensions. Na×Da data of A is an objective variable, and Nb×Db data of B is a predictor. Learning is performed using the objective variable and the predictor, using the Sparse modeling as an example of the learning model. In the Sparse modeling, a regularization algorithm may be used such as the LASSO algorithm or Elastic Net, with which over-training can be avoided and a highly accurate predictor is selected, for example.
(3) The process in (2) is executed Da×Na times. With this, a model (inference formula) for estimating a signal A from a signal B is generated.
(4) Using the model (first learning model) that has learned in (3), an EEG signal on the scalp is estimated based on an EEG signal measured in the ear canals, even when an EEG signal on the scalp is not used (signal A is not present).

### SPECIFIC EXAMPLES

Next, the inventors have performed the following specific experiments. A user that has worn the earphone set 10 and the headgear 20 as shown in FIG. 1 was asked to think of five target commands, which are up (↑) down (↓) left right (→) and stop, for 12 seconds each.

Here, the signal A on the scalp is measured using electrodes (e.g., T7, T8, Cz) in the headgear 20. At the same time, the EEG signal B in the ear canals is acquired using left and right electrodes of the earphone 100. The acquired EEG signals A and B are transformed into power spectra, and thereafter learning and estimation is performed by performing the processing (1) to (4).

Next, learning is performed using 80% of data of the EEG signal acquired from the electrodes on the scalp, the EEG signal acquired from the electrodes in the ear canals, and the EEG signal on the scalp estimated in (4) described above from the EEG signal acquired from the electrodes in the ear canals. Furthermore, models (second learning models) generated for the respective five commands are applied to the rest of 20% of data that has not been used for learning, and the command for which the likelihood of estimation is highest is the estimation result. The estimation accuracy of the estimation result is shown below.

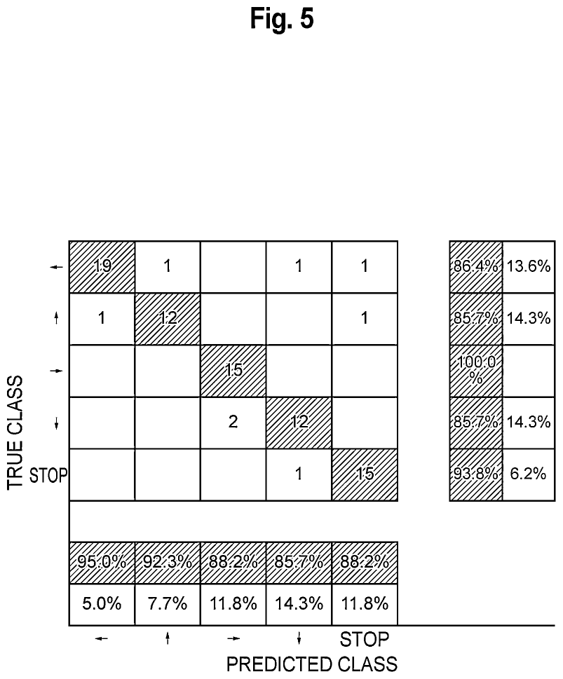

FIG. 5 is a diagram illustrating the estimation accuracy when learning EEG signals on the scalp. In the example shown in FIG. 5, the EEG signal is measured using three electrodes (T7, T8, Cz) as the electrodes on the scalp. Here, supervised learning is performed using 80% of data of the measured EEG signals as learning data, and the command is estimated using the rest of 20% of data as input data. In this learning (the same applies below), the command actually imaged by the user is treated as the correct answer label.

In the example shown in FIG. 5, the accuracy of estimated command is shown in the following result.
Number of correct answers 73, Number of incorrect answers 8
Correct answer rate 90.12%

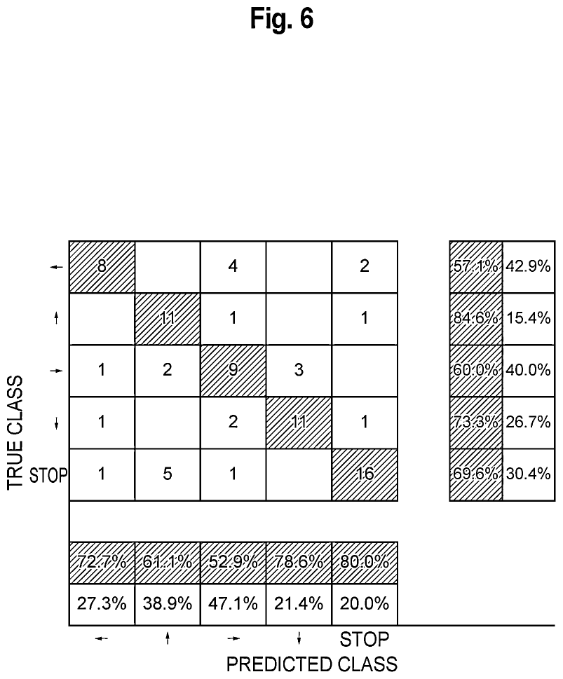

FIG. 6 is a diagram illustrating the estimation accuracy when learning EEG signals in the ear canals. In the example shown in FIG. 6, the EEG signals are measured using two electrodes in total, which are one each of left and right electrodes of the ear canals. Here, supervised learning is performed using 80% of data of the measured EEG signals as learning data, and the command is estimated using the rest of 20% of data as input data.

In the example shown in FIG. 6, the accuracy of estimated command is shown in the following result.
Number of correct answers 55, Number of incorrect answers 25
Correct answer rate 68.75%

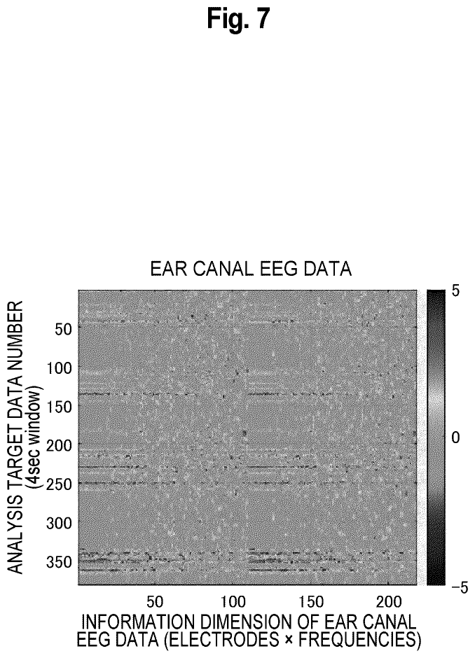

FIG. 7 is a diagram illustrating an example of the model of EEG signals in the ear canals. The vertical axis shown in FIG. 7 shows data numbers of analysis target when using a four second-moving window. The horizontal axis shows the information dimension (Nb (number of electrodes=2)×Db (dimension of specific frequencies)) of the EEG signal in the ear canals. The model (second learning model) shown in FIG. 7 is obtained by performing supervised learning using 80% of data as learning data, and is generated for each command.

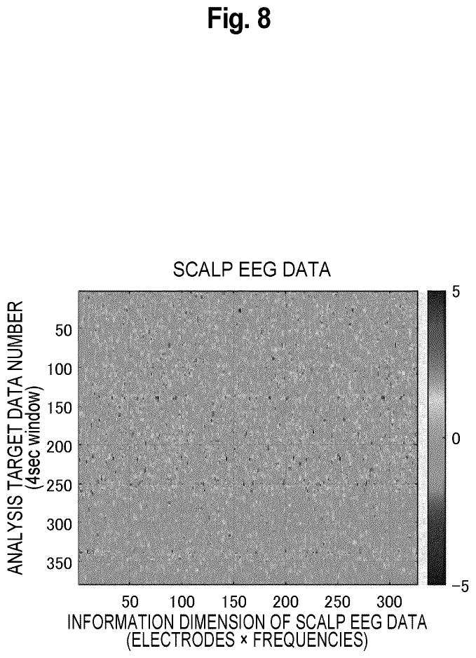

FIG. 8 is a diagram illustrating an example of the model of EEG signals on the scalp. The vertical axis shown in FIG. 8 shows data numbers of analysis target when using a four second-moving window. The horizontal axis shows the information dimension (Na (number of electrodes=3)×Da (dimension of specific frequencies)) of the EEG signal on the scalp. The model (second learning model) shown in FIG. 8 is obtained by performing supervised learning using 80% of data as learning data, and is generated for each command.

Here, the EEG signal on the scalp is estimated from an EEG signal in the ear canals that has actually measured, using the first learning model generated by performing the processing (1) to (4) described above.

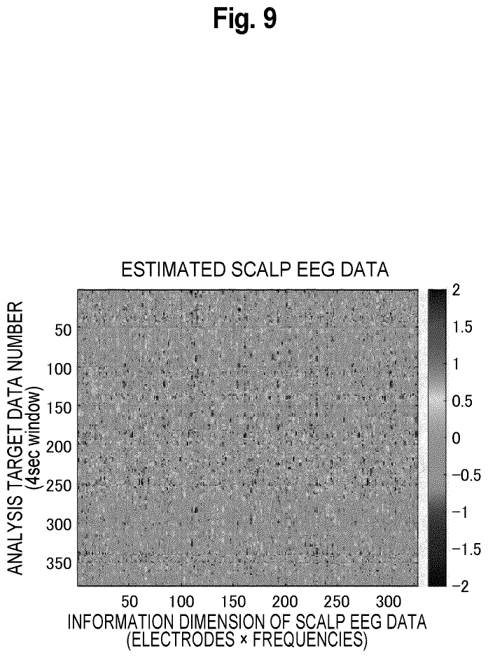

FIG. 9 is a diagram illustrating an example of the model of the estimated EEG signal on the scalp. In the example shown in FIG. 9, the aforementioned first learning model is generated for each dimension of electrodes (number of measurement positions)×frequencies, an estimated EEG signal of the scalp is generated, and 0.6739 is obtained as the correlation between the estimated values and the measured values for all dimensions. Therefore, in this experiment, it is understood that the correlation between the EEG signal on the scalp estimated from the EEG signal in the ear canals and the actual EEG signal on the scalp is about 0.7.

From the above, with respect to the EEG signal on the scalp, there is some correlation between the measured values and the estimated values, and therefore, the inventors has generated the second learning models using estimated values of the EEG signal on the scalp, and studied the accuracy of the command estimated using an estimated value of the EEG signal on the scalp.

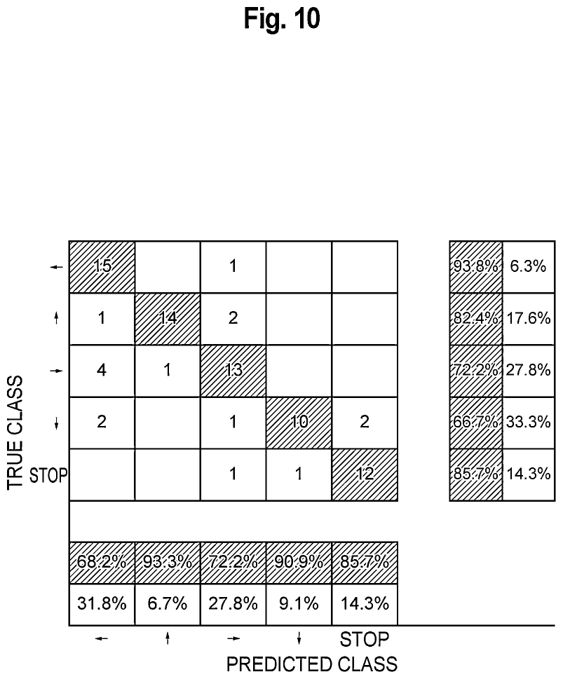

FIG. 10 is a diagram illustrating the estimation accuracy when learning estimated EEG signals on the scalp. In the example shown in FIG. 10, supervised learning is performed using, as learning data, 80% of data of estimated values of the EEG signal on the scalp estimated from the measured values of the EEG signal in the ear canals, and the command is estimated using the remaining 20% of data as input data.

In the example shown in FIG. 10, the accuracy of estimated command is as shown in the following result.
Number of correct answers 64, Number of incorrect answers 16
Correct answer rate 80.00%

Therefore, by using a learning model that has learned using estimated EEG signals on the scalp as learning data, the correct answer rate has increased from 68.75% of the EEG signal in the ear canals to 80%. That is, the estimation accuracy of command has increased when a learning model is used that has learned estimated values of the EEG signals on the scalp that are estimated from the EEG signals in the ear canals, relative to the case of using measured values of the EEG signals in the ear canal as learning data. Accordingly, even in a case where the EEG signal in the ear canals is measured, as a result of estimating the command after estimating the EEG signal on the scalp from the EEG signal in the ear canals, the command (an example of the brain activity) can be predicted with a certain degree of accuracy.

From the above, by integrating an EEG signal in the ear canals and an estimated EEG signal on the scalp as a vector, and using both of the EEG signals, the brain state (intention and emotion) of a user can be estimated at a higher accuracy, and both of the EEG signals can be used for neurofeedback. Also, the EEG signal can be acquired from the ear canals, measurement need not be performed using a brain activity measuring instrument such as a headgear as in a known technique, and therefore the EEG signal can be easily acquired, and also the estimation accuracy of brain activity can be improved even with a limited EEG signal.

Also, the fact that the relationship is present between the EEG signal in the ear canals and the EEG signal on the scalp has been found out as described above, and therefore it can be said that, not limited to the EEG acquired from the scalp, similar result can be obtained regarding a brain activity that is measured using a predetermined brain activity measuring instrument. This is because, even though the EEG may differ depending on the measurement position, the EEG that originally occurs is the same, and there is a correlation between the EEG measured by a brain activity measuring instrument and the EEG in the ear canals, regardless of the brain activity measuring instrument to be used. Here, the predetermined brain activity measuring instrument includes apparatuses that can measure brain activity such as a measuring instrument using scalp electrodes described above, a measuring instrument that measures brain activity using electrodes in the cranium, a measuring instrument that measures brain activity using the functional magnetic resonance imaging (fMRI), and a measuring instrument that measures brain activity using the near-infrared spectroscopy (NIRS). The apparatuses that acquire EEG information measured in the ear canals and EEG information measured by another brain activity measuring instrument, and generate and utilize the learning models will be described below.

### Exemplary Configuration of Server

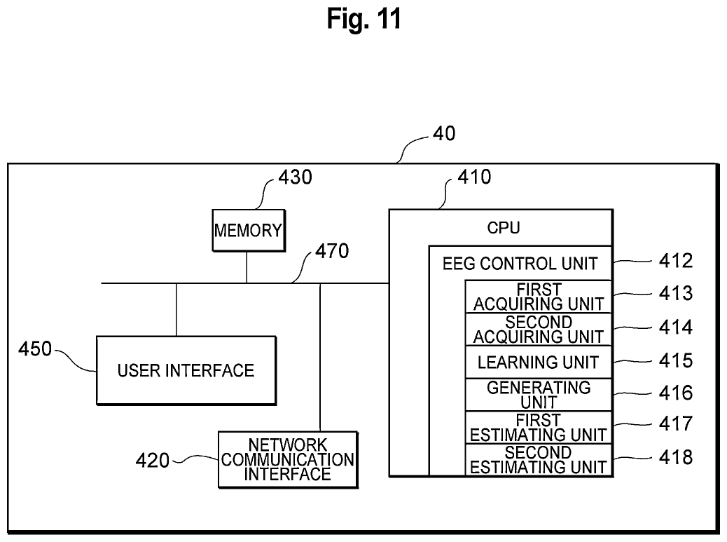

FIG. 11 is a block diagram illustrating an example of the information processing apparatus 40 according to the embodiment. The information processing apparatus 40 is a server, for example, and may also be configured by one or more apparatuses. Also, the information processing apparatus 40 processes EEG information, and analyzes the brain activity (a user images, or the like) from EEG information using a learning function of artificial intelligence (AI), for example. The information processing apparatus 40 may also be denoted as a server 40.

The server 40 includes at least one processing apparatus (processor: CPU (Central Processing Unit)) 410, at least one network communication interface 420, a memory 430, a user interface 450, and at least one communication bus 470 for connecting these constituent elements to each other.

The server 40 may also include a user interface 450 depending on the case, and the user interface 450 may include a display apparatus (not shown), and a keyboard and/or a mouse (or another input apparatus such as a pointing device (not shown)).

The memory 430 is a high-speed random access memory such as a DRAM, an SRAM, a DDR RAM, or another random access solid-state storage apparatus, or may also be at least one nonvolatile memory such as a magnetic disk storage apparatus, an optical disk storage apparatus, a flash memory device, or another nonvolatile solid-state storage apparatus, for example.

Also, another example of the memory 430 may include at least one storage apparatus that is installed in a place remote from the CPU 410. In one embodiment, the memory 430 stores a following program, module and data structure, or subsets thereof.

The at least one processing apparatus (CPU) 410 reads out a program from the memory 430, and executes the program as needed. For example, the at least one processing apparatus (CPU) 410 may configure an EEG control unit 412, a first acquiring unit 413, a second acquiring unit 414, a learning unit 415, a generating unit 416, a first estimating unit 417, and a second estimating unit 418, by executing programs stored in the memory 430. The EEG control unit 412 controls and processes EEG information that is acquired successively, and controls the following sets of processing.

The first acquiring unit 413 acquires first EEG information (e.g., an EEG signal) that is measured by first bio-electrodes that come into contact with the ear canals of a predetermined user. For example, the user is asked to wear the earphone set 10 as shown in FIGS. 3 and 4, and the ear chips (e.g., first bio-electrodes) of the earphone 100 come into contact with the ear canals, and as a result, the first EEG information obtained by sensing performed by the ear chips, which are elastic electrodes, is acquired.

The second acquiring unit 414 acquires second EEG information that is measured on the predetermined user by a predetermined brain activity measuring instrument. As an example of the predetermined brain activity measuring instrument, a brain activity measuring instrument that performs measurement using second bio-electrodes on a scalp is used. The second acquiring unit 414 acquires second EEG information (e.g., an EEG signal) that is measured by the brain activity measuring instrument. For example, the second acquiring unit 414 acquires the second EEG information at the same timing as the first acquiring unit 413. Note that the EEG information is information generated according to the brain activity measuring instrument, and may be image information indicating brain activity, for example.

The learning unit 415 learns the relationship between a first feature of the first EEG information and a second feature of the second EEG information. For example, the learning unit 415 performs machine learning in order to find out the relationship between the two pieces of EEG information, using the first EEG information and the second EEG information as learning data. Also, if the relationship between the first EEG information and the second EEG information can be extracted without performing learning, the learning unit 415 may also use this method.

The generating unit 416 generates a learning model (first learning model) that has learned the relationship. For example, when the learning unit 415 has performed learning with respect to learning data including the first and second EEG information, the generating unit 416 constructs and generate the first learning model based on the learning result.

Accordingly, using the EEG information in the ear canals and the EEG information measured by a predetermined brain activity measuring instrument, the two pieces of information having been measured at the same timing, the relationship between features is caused to learn, and as a result, a model for estimating, from the EEG information in the ear canals, EEG information from which a wide range of brain information can be acquired can be constructed and generated.

The first estimating unit 417 estimates, at a timing (in an estimation phase) different from the learning phase in which a first learning model is constructed, fourth EEG information corresponding to EEG information measured by the predetermined brain activity measuring instrument using third EEG information measured using the first bio-electrodes and the constructed first learning model. For example, the first estimating unit 417 estimates the fourth EEG information corresponding to the EEG information to be measured by the predetermined brain activity measuring instrument by inputting the third EEG information acquired in the estimation phase to the first learning model (inference formula including appropriate parameters) generated by the learning unit 415 and the generating unit 416.

Accordingly, by using the first learning model that is appropriately constructed, the EEG information from which a wide range of brain information can be acquired can be estimated from EEG information that is measured as a result of electrodes provided in ear chips of an earphone or the like coming into contact with the ear canals.

The second estimating unit 418 estimates brain activity information (e.g., information regarding a predetermined event imaged by a user) based on fourth EEG information to be estimated. For example, the learning unit 415 may construct a learning model (second learning model) for each brain activity information, by performing supervised learning in which the brain activity information is the correct answer label, using learning data including the fourth EEG information, in the stage of learning phase. The brain activity information may be a command (a command imaged by the user) used in the experiment described above, or may also be information such as predetermined emotion (delight, anger, sorrow and pleasure, etc.), a predetermined action (run, walk, weep, etc.), and a predetermined thing (food, animal, plant, etc.).

The second estimating unit 418 estimates the brain activity information by inputting EEG information estimated from the third EEG information that is successively acquired from the first bio-electrodes to the second learning models that have learned using the fourth EEG information for respective pieces of brain activity information. The second estimating unit 418 determines the brain activity information related to a second learning model with which the highest likelihood has been obtained as the estimation result.

Accordingly, EEG information from which a wide range of brain information can be acquired is estimated from EEG information in the ear canals, and by using learning models that have learned using the EEG information as learning data, the brain activity information can be more appropriately estimated compared with the case where only the EEG information in the ear canals is used.

Also, the learning unit 415 may, when performing learning of the first learning model, set the first EEG information as a predictor and the second EEG information as an objective variable, and perform the Sparse modeling. For example, the learning unit 415 uses, when performing machine learning, the Sparse modeling in which the relationship between the two pieces of EEG information is focused on, and as a result, a factor can be selected that is closely related between the first feature of the first EEG information and the second feature of the second EEG information.

Accordingly, as a result of the learning unit 415 using the Sparse modeling in machine learning, the relationship between the two pieces of EEG information can be accurately extracted, and the EEG information to be measured by the brain activity measuring instrument can be appropriately estimated based on this relationship.

Also, the objective variable may be represented by information (matrix or vector representation) of Na (number of measurement positions of the brain activity measuring instrument)×Da (dimension of specific frequencies), and the predictor may be represented by information (matrix or vector representation) of Nb (number of measurement positions of first bio-electrodes)×Db (dimension of specific frequencies). The number of measurement positions of the brain activity measuring instrument is larger than the number of measurement positions of the first bio-electrodes (Na>Nb), and the Sparse modeling is used in order to prevent this positional different from influencing the analysis.

Accordingly, the objective variable and the predictor can be appropriately set, and the machine learning can be appropriately performed, and as a result, the EEG information to be measured by the brain activity measuring instrument can be appropriately estimated from the EEG information in the ear canals.

As described above, according to the information processing apparatus 40, a model (first learning model) can be generated for estimating the EEG information to be measured by the brain activity measuring instrument from the EEG information in the ear canals. Also, the information processing apparatus 40 can construct models (second learning models) with which the estimation accuracy can be improved by using, when the brain activity information is estimated from EEG information in the ear canals, the estimated EEG information as learning data.

### Exemplary Configuration of Processing Terminal

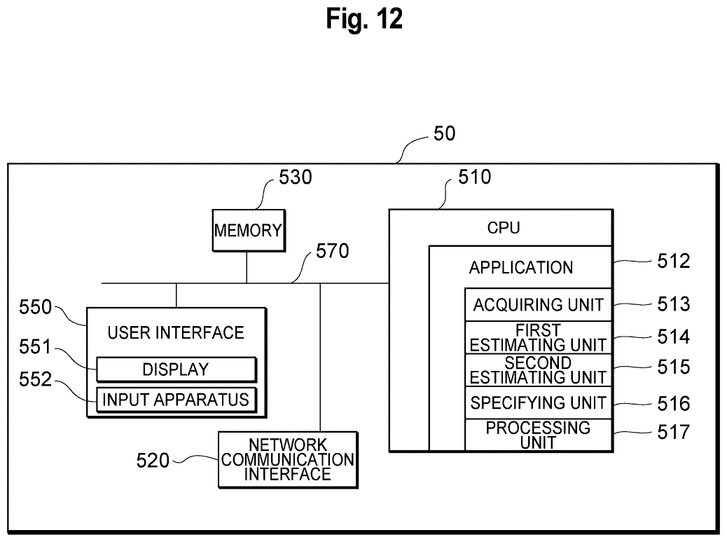

FIG. 12 is a block diagram illustrating an example of the information processing apparatus 50 according to the embodiment. The information processing apparatus 50 is a smartphone, a mobile phone (feature phone), a computer, a tablet terminal, or a PDA (Personal Digital Assistant), for example. The information processing apparatus 50 may also be denoted as a processing terminal 50.

The processing terminal 50 includes at least one processing apparatus (CPU) 510, at least one network communication interface 520, a memory 530, a user interface 550, and at least one communication bus 570 for connecting these constituent elements to each other.

The user interface 550 includes a display apparatus 551 and an input apparatus (keyboard and/or mouse, or any other pointing device, etc.) 552. Also, the user interface 550 may also be a touch panel.

The memory 530 is a high-speed random access memory such as a DRAM, an SRAM, a DDR RAM, or another random access solid-state storage apparatus, or may also be at least one nonvolatile memory such as a magnetic disk storage apparatus, an optical disk storage apparatus, a flash memory device, or another nonvolatile solid-state storage apparatus, for example.

Also, another example of the memory 530 may include at least one storage apparatus that is installed in a place remote from the CPU 510. In one embodiment, the memory 530 stores a following program, module and data structure, or subsets thereof.

The at least one processing apparatus (CPU) 510 reads out a program from the memory 530, and executes the program as needed. For example, the at least one processing apparatus (CPU) 510 may configure an application 512 by executing a program stored in the memory 530. This application 512 is an application for processing an EEG, and includes an acquiring unit 513, a first estimating unit 514, a second estimating unit 515, a specifying unit 516, and a processing unit 517.

The acquiring unit 513 acquires EEG information measured by first bio-electrodes that come into ear canals of a predetermined user. For example, as a result of the first bio-electrodes provided in ear chips of an earphone coming into contact with ear canals with pressure, the EEG information is measured. The acquiring unit 513 receives and acquires EEG information measured by the earphone.

The first estimating unit 514 estimates, using a first learning model generated by the information processing apparatus 40 described above, another EEG information corresponding to the EEG information measured by the brain activity measuring instrument, from the EEG information acquired by the acquiring unit 513. For example, the first learning model is a model that has learned the relationship between a first feature of first EEG information measured by the first bio-electrodes that come into contact with the ear canals of the predetermined user and a second feature of second EEG information measured by the predetermined brain activity measuring instrument with respect to the predetermined user. That is, the first estimating unit 514 estimates, using the first learning model acquired from the information processing apparatus 40, another EEG information to be measured by the brain activity measuring instrument from the EEG information in the ear canals.

The second estimating unit 515 estimates brain activity information based on the other EEG information estimated by the first estimating unit 514. For example, the second estimating unit 515 acquires second learning models for respective pieces of brain activity information from the information processing apparatus 40, and estimates the brain activity information of the user at the time of measurement, by inputting the EEG information estimated by the first estimating unit 514 into the second learning models that have learned using the other EEG information. The second estimating unit 515 determines the brain activity information related to a second learning model with which highest likelihood has been obtained as the estimation result.

The specifying unit 516 specifies the command based on the brain activity information estimated by the second estimating unit 515. For example, the specifying unit 516 may associate each piece of brain activity information with the corresponding command, and specify the command corresponding to the estimated brain activity information. The commands are operation commands (button pressing down, etc.) when operating the processing terminal 50, and operation commands (reproduce, stop, halt, fast-forward, rewind, etc.) to a predetermined application (music and moving image reproduction application), for example. The brain activity information indicates that the user images up, down, right, or left, or directly images one of the aforementioned operation commands, for example.

The processing unit 517 executes the processing corresponding to the specified command. For example, the processing unit 517 executes, if the command is a volume up command, processing for increasing the volume of the processing terminal 50, and if the command is a command for stopping a moving image, processing for stopping the moving image that is being reproduced by the processing terminal 50.

Accordingly, the processing terminal 50 can execute, using a command specified by the EEG information of the ear canals measured by the earphone, the processing corresponding to this command. As described above, as a result of expanding the learning data using the EEG information estimated using EEG information in the ear canals, the brain activity can be appropriately estimated, and therefore the user can operate the processing terminal 50 as desired to some degree.

Also, the acquiring unit 513 may acquire myoelectricity information (e.g., myoelectricity signal) based on a signal measured by the first bio-electrodes that come into contact with the ear canals. When the user masticates, the acquiring unit 513 can acquire myoelectricity information regarding the musculus temporalis and masseter muscle that are close to the ear canals. For example, by performing predetermined filtering processing on the signal measured from the ear canals, the myoelectricity signal can be acquired. The filtering processing may be executed on the earphone side, or may also be executed on the processing terminal 50 side.

In this case, the specifying unit 516 may also specify the command further based on the myoelectricity information acquired by the acquiring unit 513. For example, the specifying unit 516 may specify the command based on the following conditions.

Conditions: Command
Masticate once: Reproduce
Masticate twice: Fast-forward
Masticate three times: Rewind
Gnash teeth: Volume up
Masticate once and gnash teeth: Volume down
Image heart: Register in bookmark

With this, a device can be simply operated utilizing not only the EEG information but also the myoelectricity information. Also, by combining the myoelectricity information and the EEG information, abundant interfaces can be provided.

Note that, in the example shown in FIG. 12, a configuration in which the first estimating unit 514 and second estimating unit 515 are provided in the processing terminal 50 has been described, but when the first estimating unit 417 and second estimating unit 418 are provided in the server 40, as shown in FIG. 11, the acquiring unit 513 may acquire estimated brain activity information, and the specifying unit 516 may specify the command based on the brain activity information acquired by the acquiring unit 513. In this way, the processing may be distributed between the server 40 and processing terminal 50, as appropriate, and the sets of processing in which the respective functions are provided may be determined depending on the implementation.

<Operations>

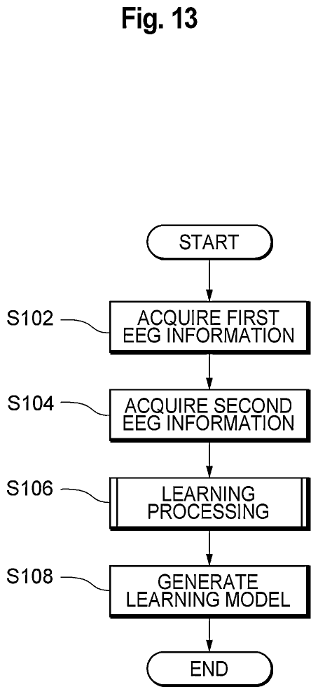

Next, the operation of the system 1 according to the embodiment will be described. FIG. 13 is a flowchart illustrating an example of model generation processing of the server 40 according to the embodiment. In the example shown in FIG. 13, an example in which the server 40 generates a learning model will be described.

In step S102, the first acquiring unit 413 acquires first EEG information measured by the first bio-electrodes that come into contact with the ear canals of a predetermined user. For example, the first acquiring unit 413 acquires EEG information measured by the bio-electrodes provided in the ear chips of an earphone.

In step S104, the second acquiring unit 414 acquires second EEG information that is measured for the predetermined user by a predetermined brain activity measuring instrument at the same timing as the first EEG information. For example, the second acquiring unit 414 directly or indirectly acquires EEG information measured by electrodes that are set on the scalp based on the ten-twenty electrode system of the International Federation. The sequence of steps S102 and S104 is not specifically limited, and two pieces of EEG information that are measured at the same time may also be acquired at the same time.

In step S106, the learning unit 415 learns the relationship between a first feature of the first EEG information and a second feature of the second EEG information. For example, the learning unit 415 finds out the relationship between the first EEG information and the second EEG information by learning the two pieces of EEG information using a predetermined learning model.

In step S108, the generating unit 416 generates a learning model that has learned the relationship. For example, the generating unit 416 constructs and generates a model (first learning model) of inference formula representing the relationship between the two pieces of EEG information such that the second EEG information is estimated from the first EEG information, by the learning unit 415 performing learning of the two pieces of EEG information.

According to the processing described above, by performing learning of the relationship between the feature of the EEG information in the ear canals and the feature of the EEG information measured by the brain activity measuring instrument that are measured at the same timing, using these pieces of EEG information, for example, a model for estimating the EEG information from which a wide range of brain information can be acquired from the EEG information in the ear canals can be constructed and generated.

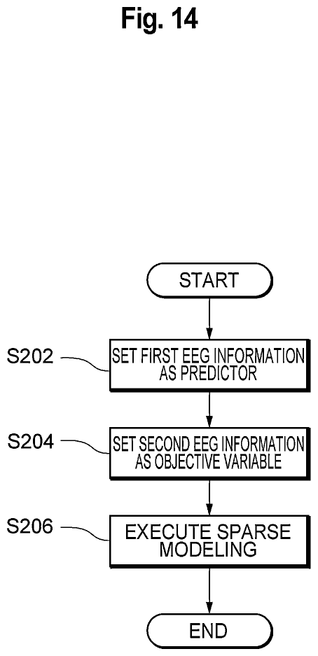

FIG. 14 is a flowchart illustrating an example of the learning processing according to the embodiment. In the example shown in FIG. 14, an example in which the server 40 applies the Sparse modeling when generating the first learning model will be described, but the model may also be generated using another classification model.

In step S202, the learning unit 415 sets the first EEG information as a predictor. For example, the learning unit 415 sets information (may be a matrix or vector representation) of Nb (number of measurement positions of the first bio-electrode)×Db (dimension of specific frequencies) as the predictor.

In step S204, the learning unit 415 sets the second EEG information as an objective variable. For example, the learning unit 415 sets information (may be a matrix or vector representation) of Na (number of measurement positions of the brain activity measuring instrument)×Da (dimension of specific frequencies) as the objective variable.

In step S206, the learning unit 415 executes machine learning utilizing the Sparse modeling using the set objective variable and predictor.

As a result of the processing described above, the learning unit 415 uses, when performing machine learning, the Sparse modeling in which the relationship between the two pieces of EEG information is focused on, and as a result, a factor can be selected that is closely related between the first feature of the first EEG information and the second feature of the second EEG information. As a result of using the Sparse modeling for machine learning, the learning unit 415 can accurately extract the relationship between two pieces of EEG information, and can appropriately estimate the EEG information to be measured by the brain activity measuring instrument based on this relationship.

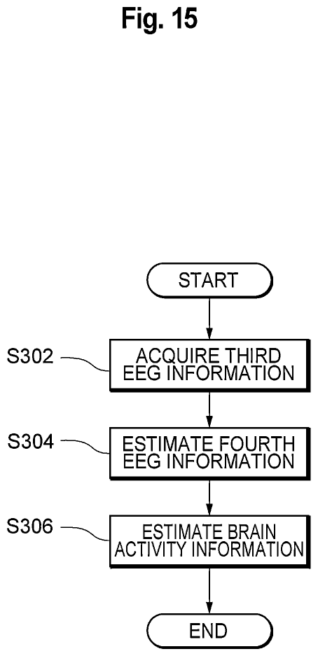

FIG. 15 is a flowchart illustrating an example of brain activity estimation processing according to the embodiment. In the example shown in FIG. 15, the processing in which the server 40 estimates the brain activity information will be described, but when the processing is performed by another apparatus as well, the processing is basically the same.

In step S302, the first estimating unit 417 acquires the EEG information (third EEG information) in the ear canals that is measured by the first bio-electrodes in an estimation phase.

In step S304, the first estimating unit 417 estimates fourth EEG information corresponding to the EEG information to be measured by the brain activity measuring instrument using the third EEG information to be acquired and the generated first learning model. For example, the first estimating unit 417 estimates the fourth EEG information corresponding to the EEG information to be measured by the brain activity measuring instrument by inputting the third EEG information acquired in the estimation phase to the first learning model (inference formula including appropriate parameters) generated by the learning unit 415 and the generating unit 416.

In step S306, the second estimating unit 418 estimates the brain activity information regarding the user based on the fourth EEG information estimated by the first estimating unit 417. For example, the second estimating unit 418 estimates the brain activity information by inputting the fourth EEG information estimated from the third EEG information that is successively acquired into the second learning models that have performed supervised learning in which brain activity information is the correct answer label using the fourth EEG information.

As a result of the processing described above, EEG information from which a wide range of brain information can be acquired is estimated from the EEG information in the ear canals, and by using the learning models that have learned using the EEG information as learning data, the brain activity information can be more appropriately estimated compared with the case where only the EEG information in the ear canals is used.

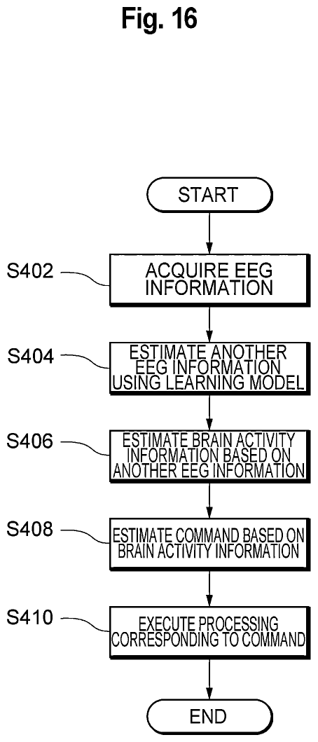

FIG. 16 is a flowchart illustrating an example of command operation processing according to the embodiment. In the example shown in FIG. 16, the processing terminal 50 acquires EEG information in the ear canals and estimates a wide range of EEG information, but a configuration may also be adopted in which the processing terminal 50 acquires the wide range of EEG information estimated by the server 40.

In step S402, the acquiring unit 513 acquires EEG information measured by the first bio-electrodes that come intact with the ear canals of the predetermined user. For example, the acquiring unit 513 receives and acquires EEG information measured by the earphone.

In step S404, the first estimating unit 514 estimates another EEG information corresponding to the EEG information to be measured by the brain activity measuring instrument, from the EEG information acquired by the acquiring unit 513 using the first learning model generated by the server 40 described above.

In step S406, the second estimating unit 515 estimates brain activity information regarding the predetermined user based on the other EEG information estimated by the first estimating unit 514. For example, the second estimating unit 515 acquires the second learning models from the server 40, and estimates the brain activity information of the user by inputting the EEG information estimated by the first estimating unit 514 into the second learning models that have learned using the other EEG information.

In step S408, the specifying unit 516 specifies a command based on the brain activity information estimated by the second estimating unit 515. For example, the specifying unit 516 may associate each piece of brain activity information with the corresponding command, and specify the command corresponding to the estimated brain activity information.

In step S410, the processing unit 517 executes the processing corresponding to the specified command. For example, the processing unit 517 executes, if the command is a volume up command, processing for increasing the volume of the processing terminal 50, and if the command is a command for stopping a moving image, processing for stopping the moving image that is being reproduced by the processing terminal 50.

According to the processing described above, the processing terminal 50 can execute, using a command specified by the EEG information of the ear canals measured by the earphone, the processing corresponding to this command. As described above, as a result of expanding the learning data using the wide range of EEG information estimated using EEG information in the ear canals, the brain activity can be appropriately estimated, and therefore the user can operate the processing terminal 50 as desired to some degree.

### APPLICATION EXAMPLES

As an interface in which the above-described EEG information is used, a hand-free operation is required for controlling the interface without using hands, when utilizing devices such as a smartphone and VR (Virtual Reality) and AR (Augmented Reality) devices. In this case, by using the embodiment described above, the device can be simply operated using EEG information (or myoelectricity information that can be acquired), using an earphone that acquires EEG information. For example, when a user masticates, a myoelectricity signal of musculus temporalis and masseter muscle that are close to the ear canals is acquired from the electrodes of the earphone, and EEG information at the time when the user images a specific image can be acquired from the electrodes of the earphone. Also, as a result of applying the embodiment described above, a device can be operated using the EEG information and myoelectricity information that are acquired from electrodes in the ear canals.

As described above, the embodiment described above is merely an example for describing the technique of the present disclosure, and is not intended to limit the technique of the present disclosure to the embodiment, and the technique of the present disclosure can be variously modified without departing the gist thereof.

In the embodiment described above, a wider range of EEG information is estimated from the EEG information acquired from ear canals, but the information to be estimated is not limited to the wide range of EEG information. For example, biological information that can be acquired from a human body can also be estimated from EEG information or biological information that can be acquired from ear canals. As a specific example, the biological signals that can be acquired from a head or the vicinity thereof include an ocular potential signal, a heart-rate signal from arteria carotis or the like, and a myoelectricity signal from masseter muscle or musculus temporalis, in addition to the EEG. Also, the biological signals that can be acquired from a human body include an electrocardiogram signal, for example. These pieces of biological information may be measured using a biosensor (biological information measuring instrument) for measuring the corresponding biological information.

A second acquiring unit 414 in the modification described above acquires the biological information described above instead of the second EEG signal, and the learning unit 415 learns the relationship between a first feature of first EEG information in the ear canals and a third feature of a biological signal. A generating unit 416 generates a learning model (third learning model) that has learned this relationship. A first estimating unit 417 estimates biological information using third EEG information and the third learning model. A second estimating unit 418 estimates activity information regarding the human body using the estimated biological information. For example, if the biological information is an ocular potential signal, nictation, a line of sight movement, and sleepiness can be estimated, if the biological information is a myoelectricity signal, the motion at that part can be estimated, if the biological information is a heart-rate signal, an excited state or a state of tension can be estimated, and if the biological information is an electrocardiogram signal, the cardiac condition can be estimated. In the estimation performed by the second estimating unit 418, a learning model may be used that has been constructed by performing supervised learning in which the biological signals are learning data, and the corresponding states are correct answer labels. Also, at least one of the sets of processing described in the server 40 and the processing terminal 50 may be performed by another apparatus in order to distribute loads. For example, the processing units (first acquiring unit 413, second acquiring unit 414, and learning unit 415) in the learning phase may be provided in the server, and the processing units (first estimating unit 417 and second estimating unit 418) in the estimation phase may be provided in a user terminal. Here, necessary data is transmitted and received to each other. Also, the information acquired from ear canals is not limited to the EEG information, and may be biological information such as ocular potential information or myoelectricity information. Therefore, instead of the EEG information in ear canals that is used for learning and estimation, biological information acquired from the ear canals may also be used.

### REFERENCE SIGNS LIST

1 System
10 Earphone set
20 Headgear
30, 40, 50 Information processing apparatus
100 Earphone
104 Nozzle
106 Ear chip (elastic electrode)
410 CPU
412 EEG control unit
413 First acquiring unit
414 Second acquiring unit
415 Learning unit
416 Generating unit
417 First estimating unit
418 Second estimating unit
430 Memory
510 CPU
512 Application
513 Acquiring unit
514 First estimating unit
515 Second estimating unit
516 Specifying unit
517 Processing unit
530 Memory

1. An information processing method to be executed by a processor included in an information processing apparatus, the method comprising:
acquiring first EEG information that is measured by a first bio-electrode that comes into contact with an ear canal of a predetermined user;
acquiring second EEG information that is measured by a brain activity measuring instrument with respect to the predetermined user at the same timing as the first EEG information;
learning a relationship between a first feature of the first EEG information and a second feature of the second EEG information; and
generating a learning model that has learned the relationship.

2. The information processing method according to claim 1, further comprising:
estimating fourth EEG information corresponding to the second EEG information using third EEG information measured by the first bio-electrode and the learning model.

3. The information processing method to claim 2, further comprising
estimating brain activity information regarding the predetermined user, based on the fourth EEG information.

4. The information processing method according to claim 1,
wherein the learning includes setting the first EEG information to a predictor and the second EEG information to an objective variable, and performing Sparse modeling.

5. The information processing method according to claim 4,
wherein the objective variable is represented by information of Na (number of measurement positions of the brain activity measuring instrument)×Da (dimension of specific frequencies), and the predictor is represented by information of Nb (number of measurement positions of the first bio-electrode)×Db (dimension of specific frequencies).

6. A non-transitory storage medium storing a program which, when executed by a processor included in an information processing apparatus, cause the processor to perform the following:
acquiring first EEG information that is measured by a first bio-electrode that comes into contact with an ear canal of a predetermined user;
acquiring second EEG information that is measured by a brain activity measuring instrument with respect to the predetermined user at the same timing as the first EEG information;
learning a relationship between a first feature of the first EEG information and a second feature of the second EEG information; and
generating a learning model that has learned the relationship.

7. An information processing apparatus including a processor,
the processor executing a program to perform the following:
acquiring first EEG information that is measured by a first bio-electrode that comes into contact with an ear canal of a predetermined user;
acquiring second EEG information that is measured by a brain activity measuring instrument with respect to the predetermined user at the same timing as the first EEG information;
learning a relationship between a first feature of the first EEG information and a second feature of the second EEG information; and
generating a learning model that has learned the relationship.

8. The information processing method according to claim 1, further comprising:
estimating other EEG information from the acquired EEG information by using the, learning model, the other EEG information corresponding to the second EEG information;
estimating brain activity information regarding the user, based on the other EEG information;
specifying a command, based on the brain activity information; and
executing processing corresponding to the command.

9. The information method according to claim 8,
wherein the acquiring includes acquiring myoelectricity information based on information measured by the first bio-electrode, and
the specifying includes specifying the command, based further on the myoelectricity information.

10. The non-transitory storage medium according to claim 6, wherein the program further causes the processor to execute the following:
estimating other EEG information from the acquired EEG information by using the the other EEG information corresponding to the second EEG information;
estimating brain activity information regarding the user, based on the other EEG information;
specifying a command, based on the brain activity information; and
executing processing corresponding to the command.

11. The information processing apparatus according to claim 7,
the processor further executing the following:
estimating other EEG information from the acquired EEG information by using the learning model, the other EEG information corresponding to the second EEG information;
estimating brain activity information regarding the user, based on the other EEG information;
specifying a command, based on the brain activity information; and
executing processing corresponding to the command.

12. (canceled)
